(19) United States
(12) Patent Application Publication
(10) Pub. No.: US 2004/0173401 A1
(43) Pub. Date: Sep. 9, 2004

(54) Stethoscope for mechanics to diagnose vehicle sounds

(75) Inventor: Scotty R. Kurtz, Clarinda, IA (US)

Correspondence Address: BANNER & WITCOFF, LTD., TEN SOUTH WACKER DRIVE, SUITE 3000, CHICAGO, IL, 60606, US

(73) Assignee: Lisle Corporation, Clarinda, IA

(21) Appl. No.: 10/378,790

(22) Filed: Mar. 4, 2003

(51) Int. Cl. A61B 7/02

(52) U.S. Cl. 181/131

(57) ABSTRACT

A mechanic's stethoscope includes a frustaconical funnel member for collecting the audible sounds of a vehicle system and transmitting those sounds through hollow tubes to ear pads.

### BACKGROUND OF THE INVENTION

In a principal aspect, the present invention comprises a tool for diagnostic utilization with respect to vehicles and, more particularly, to a stethoscope for diagnosing vehicle systems by detecting and analyzing the audible sounds of such systems.

Automobile mechanics often are able to determine whether a vehicle engine or other vehicle system is in need of repair, and particularly the type of repair needed, by listening to the audible sounds associated with the particular system under investigation. For example, the sound associated with valve lifters may indicate the need to replace valve seats or bearings associated with valves. A skilled mechanic often uses a mechanic's stethoscope to facilitate such diagnostic reviews. Typical of a mechanic's stethoscope is Lisle Corporation Product No. 52500 for a Mechanic's Stethoscope. The stethoscope in this circumstance is somewhat analogous to a physician's stethoscope except for the utilization of a sound chamber and thin diaphragm attached to a probe. The probe is placed on the engine, for example. Then, as the probe vibrates the diaphragm, a sound associated with the vehicle system is generated in the sound chamber and transmitted by a hollow tube to the mechanic's ears.

Such mechanic's stethoscopes are also offered by various other tool suppliers. Such stethoscopes are popular tools for use by vehicle mechanics. Nonetheless there has remained the need for a simple and yet improved stethoscope construction which may be utilized by vehicle mechanics.

### SUMMARY OF THE INVENTION

Briefly, the present invention comprises a stethoscope especially constructed for use by a mechanic. The stethoscope includes rigid tubes associated with ear pads that are designed for insertion into the mechanic's ears. A flexible tube connects from the rigid tubes to a single rigid tube which is, in turn, attached a frustaconical sound gathering chamber. The construction of the frustaconical sound gathering chamber is such that the wide planar face of the chamber at the end opening side of the chamber has a diameter which is approximately five times the diameter of the rigid tube to which the chamber is attached. Thus, the chamber acts as an acoustical amplifier which amplifies the sound and delivers the sound vibrations through a substantially reduced, uniform diameter tube to the ears of a mechanic.

Thus, it is an object of the invention to provide an improved stethoscope for use by a mechanic to analyze the audible sounds, including the sounds of leaking fluids, that may be associated with various vehicle systems.

It is a further object of the invention to provide a stethoscope for use by a mechanic which is easy to use, economical, rugged and which transmits audible sounds to a high degree of fidelity.

These and other objects, advantages and features of the invention will be set forth in the detailed description which follows.

### BRIEF DESCRIPTION OF THE DRAWING

In the detailed description which follows, reference will be made to the drawing comprised of the following figures:

FIG. 1 is a plan view of the stethoscope of the invention;

FIG. 2 is an end view of frustaconical funnel member utilized for sound gathering for the stethoscope of the invention;

FIG. 3 is a cross sectional view of the frustaconical funnel member of FIG. 2; and

FIG. 4 is an isometric view of the stethoscope of the invention.

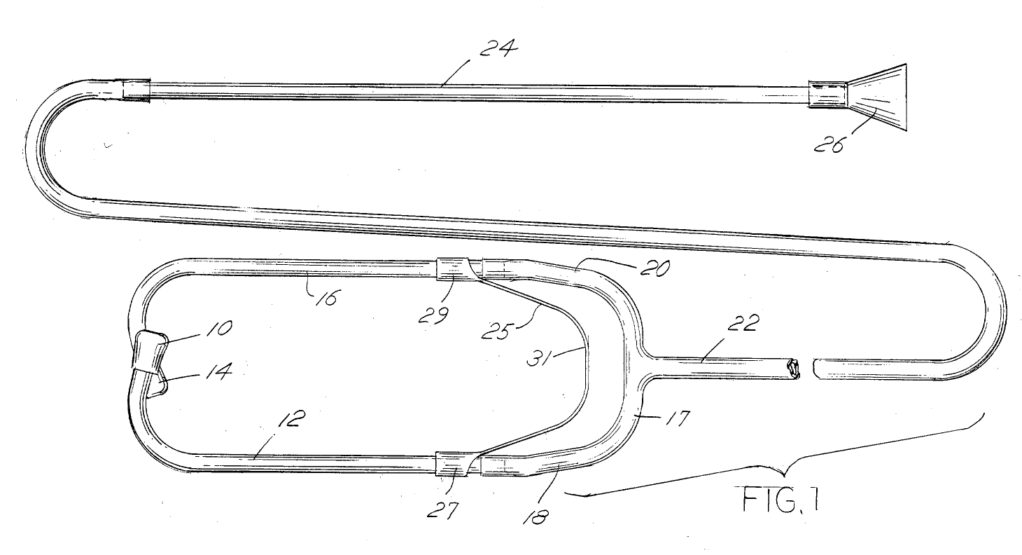

### DESCRIPTION OF THE PREFERRED EMBODIMENT

The mechanic's stethoscope of the present invention differs from prior art mechanic's stethoscopes in that prior art devices of this nature included a probe designed for placement on an engine component, for example, to sense mechanical vibrations. The mechanical vibrations were then transferred into audible sounds by vibrating a diaphragm located in a sound chamber to which a hollow tube was attached. With the device of the present invention a frustaconical horn is utilized to gather audible sounds directly. Those sounds are then transmitted through hollow tubes to the ears of a mechanic for diagnostic purposes.

Referring therefore to the figures, the stethoscope includes first ear pad 10 attached to an L-shaped hollow rigid tube 12. A second ear pad 14 is attached to a second hollow rigid L-shaped tube 16. The rigid L-shaped tubes 12, 16 connect to a flexible polymeric tubing 17 having a first bifurcated arm 18 and a second bifurcated arm 20 joined together to a single, flexible extension tube 22. The flexible extension tube 22 connects to a rigid tubular member 24, for example, a stainless steel tube 24. The stainless steel tube 24 is attached to a frustaconical sound gathering funnel member 26. A spring member 25 is attached to the spaced tubes 12, 16 for holding the tubes in a generally planar manner and for retaining the tubes 12, 16 and pads 10, 14 in position for easy placement and retention in the ears of a mechanic. The spring member 25 thus includes opposite end clips 27, 29 for attachment to legs 12, 16 respectively. Clips 27, 29 are connected by a flexible metal band 31.

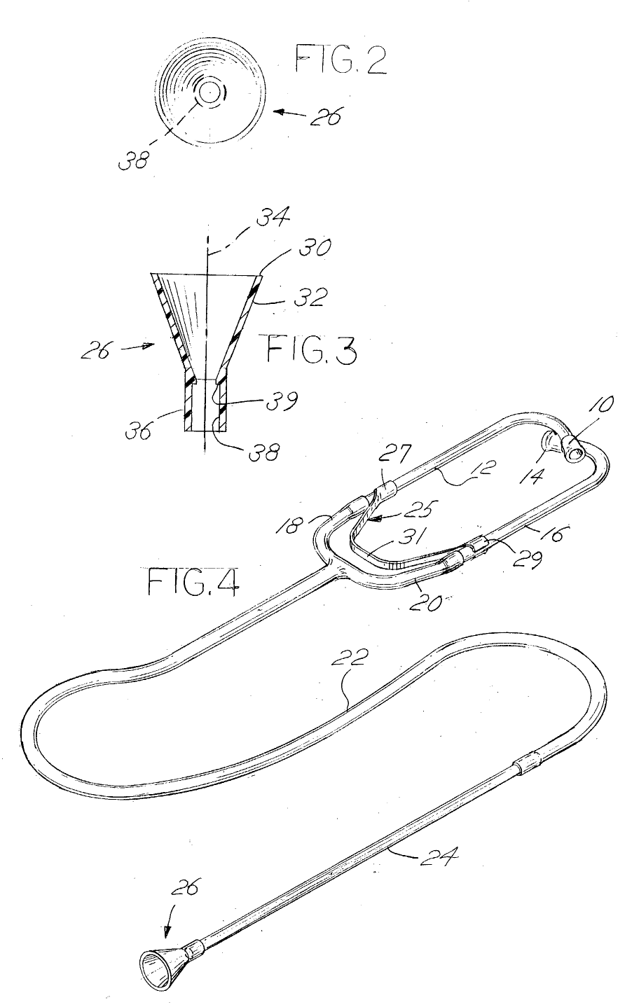

FIGS. 2 and 3 illustrate the construction of the (acoustical amplifier) sound gathering frustaconical funnel member in greater detail. The funnel member 26 includes a planar, flat outer end 30 having a width from side to side of approximately one inch. The sides of the funnel for example, side 32 converges at an angle of about 20° with respect to a centerline axis 34. The hollow frustaconical funnel member 26 connects with a stem or connection section 36 having an internal diameter of approximately 0.20 inch or approximately ⅕ of the diameter associated with the outer rim 30. The funnel member 26 further includes a recessed counterbore 38 in the connection section 36 for receipt of the rigid tube 24. Thus a land 39 is provided within the counterbore 38 for engagement with the rigid, straight, tubular member 24. The funnel member 26 is symmetrical about the axis 34. In use the planar rim 30 is positioned on an engine block, for example, or an engine head or valve cover. The mechanic then places the ear pads 10, 14 in his ears and listens to the audible sounds which are transmitted through the various hollow tubes which preferably maintain the diameter of the passage to the ear of the user at a uniform reduced diameter. This functions to maintain the benefit of acoustical amplification provided by the funnel member 26 and more particularly, the configuration of the internal chamber defined by funnel member 26.

A mechanic utilizing the stethoscope as described can thus easily and with a high degree of certainty skillfully analyze the sounds emanating from various systems in the vehicle including engine sounds, exhaust system sounds, transmission sounds, bearing sounds, drive train sounds, brake system sounds, steering and suspension system sounds and combinations thereof.

The particular configuration of the frustaconical funnel member 26 may be altered somewhat, but it is preferred that the dimensional characteristics as described be maintained including the angle of the inclination of the side wall 32 as well as the ratio of the end or rim 30 to the tube 24 in the range of 5±½ to 1. These dimensional characteristics, in the context of the vehicle analyses, have been determined to be exceedingly appropriate and important to provide for a highly accurate and sensitive diagnostic analysis. Thus, while there has been set forth a preferred embodiment of the invention, it is to be understood that the invention is limited only by the following claims and equivalents thereof.

What is claimed is:

1. A tool for diagnosing audible sounds of a vehicle said sounds selected from the group consisting of engine sounds, exhaust system sounds, transmission sounds, bearing sounds, drive train sounds, brake system sounds, steering and suspension system sounds, and combinations thereof comprising in combination:
a first straight elongate, rigid hollow tube having an outer end and an inner end;
a flexible, hollow tube having an outer end attached to the inner end of the first tube, said flexible tube including a bifurcated hollow tube at the opposite end;
a first L-shaped, rigid hollow tube having an ear pad affixed at one end and an opposite end connected to one of the bifurcated hollow tubes;
a second L-shaped, rigid hollow tube having an ear pad affixed at one end and an opposite end connected to the other of the bifurcated hollow tubes; and
a rigid funnel member having a flat planar outer rim with a first diameter, and an inner central tube extension attached to the first hollow tube to thereby provide an uninterrupted passageway from the funnel member through the ear pads, said first rigid hollow tube having a hollow tube diameter, the ratio of the first diameter of the funnel member to the hollow tube diameter is in the range of 5±½ to 1.

2. The tool of claim 1 further including a retainer spring connected to the first and second L-shaped tubes.
